(19) United States
(12) Patent Application Publication
(10) Pub. No.: US 2023/0404596 A1
(43) Pub. Date: Dec. 21, 2023

(54) DEVICES, SYSTEMS, AND METHODS FOR DEPLOYING AN IMPLANTABLE CLIP

(71) Applicants: BOSTON SCIENTIFIC SCIMED, INC., MAPLE GROVE, MN (US); Mayo Foundation for Medical Education and Research, Rochester, MN (US)

(72) Inventors: Daniel Shuey, Pine City, MN (US); Joel T. Eggert, Plymouth, MN (US); Mitchell Nelson, Hudson, WI (US); Eric Jason Krause, Big Lake, MN (US); Evan M Leingang, Plymouth, MN (US); Greg Johnson, Minneapolis, MN (US); Greg Hoepfner, Brooklyn Park, MN (US)

(73) Assignees: BOSTON SCIENTIFIC SCIMED, INC., MAPLE GROVE, MN (US); Mayo Foundation for Medical Education and Research, Rochester, MN (US)

(21) Appl. No.: 18/211,534

(22) Filed: Jun. 19, 2023

Related U.S. Application Data
(60) Provisional application No. 63/354,001, filed on Jun. 21, 2022.

(51) Int. Cl. A61B 17/128

(52) U.S. Cl. CPC A61B 17/1285; A61B 2017/00243

(57) ABSTRACT

A delivery/deployment system having a control handle configured to control delivery and/or deployment of a medical device with arms configured to grasp tissue therebetween. The system may include a device spreader with arms engaging the medical device arms and controlled by an actuator operably coupled with the control handle. The control handle may include a lever pivotable to similarly pivot the device spreader and medical device arms open or closed. A device retention assembly may be provided to retain the medical device with respect to the device spreader. The control handle may include a release element movable to shift the retention assembly into a disengaged configuration allowing release of the medical device from the device spreader. The control handle may axially translate and/or rotate the device spreader into a desired position, and may be fixable in position by a handle lock once in a desired position.

### CROSS-REFERENCE TO RELATED APPLICATIONS

This application claims the benefit of priority under 35 U.S.C. § 119 to U.S. Provisional Application No. 63/354,001, filed Jun. 21, 2022, the entire disclosure of which is hereby incorporated by reference herein for all purposes.

### FIELD

The present disclosure relates generally to the field of medical devices and systems. More particularly, the present disclosure relates to devices, systems, and methods for deploying an implantable device, such as a clip or clamp.

### BACKGROUND

Devices, systems, and methods for delivering and/or deploying medical devices with minimally invasive techniques, such as percutaneously and/or transluminally, are desirable for avoiding more complex and invasive open surgical procedures. Various techniques which do not require open surgery utilize systems and devices with various flexible elongate members capable of navigating to an anatomical site within the body from a small insertion opening in a patient's body or through a natural opening; transluminally through the body (such as through the vascular system); and to an anatomical site. To reach an anatomical site within the body, various elongate members must be steerable to navigate through tortuous pathways within the body leading to the anatomical site. Improvements to handles for steering elongate members would be welcome in the art. Moreover, to deploy an implantable device inserted percutaneously and/or transluminally, the delivery/deployment system must provide proper control of movement of the implantable device as well as proper control of any system for delivering and deploying the implantable device within the patient's body. Improvements to handles for delivering and deploying an implantable device, and/or adjusting any further devices operatively associated therewith, would be welcome in the art.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

Disclosed herein is a delivery/deployment system is configured for delivering and/or deploying a medical device having a first arm and a second arm shiftable between a closed configuration with the arms adjacent each other and an open configuration with the arms shifted apart from each other. In accordance with various principles of the present disclosure, the delivery/deployment system includes a control handle having a control handle housing and an actuator controller movable with respect to the control handle housing; a device spreader configured to deliver and deploy the medical device and having a first arm configured to engage a first arm of the medical device and a second arm configured to engage the second arm of the medical device; and an actuator coupled to the spreader device and extending to and coupled to the actuator controller. In some aspects, the actuator controller includes a lever handle pivotable with respect to the control handle housing to cause the actuator to shift the device spreader arms between closed and open configurations to shift the medical device between closed and open configurations.

In some embodiments, the actuator is coupled to the first arm of the device spreader and extends distally therefrom and then proximally to the actuator controller, and is guided around pulleys to be coupled with the lever handle.

In some embodiments, the system further includes a grasper limiter operatively associated with the control handle to limit the extent to which the device spreader opens the medical device. In some embodiments, the grasper limiter includes a limit stop movable with respect to the lever handle to limit the extent to which the lever handle is movable to cause the actuator to shift the device spreader to an open configuration. In some embodiments, the grasper limiter includes an adjustment gauge movable with respect to the control handle housing to indicate the degree to which the lever handle may open the device spreader to open the medical device.

In some embodiments, the actuator controller shifts the actuator from a neutral position to an actuated position, the system further comprising a spring assist within the control handle housing configured to assist the actuator to return to the neutral position upon releasing the lever handle. In some embodiments, the system further includes an actuator guide housing through which the actuator passes to extend to the actuator controller, and the spring assist includes a spring and a spring plate within the actuator guide housing, the spring plate being coupled with respect to the actuator, and the spring biasing the spring plate to return the actuator to the neutral position. In some embodiments, the spring assist comprises a resilient actuator guide housing configured to return the actuator to the neutral position.

In some embodiments, the device spreader includes a movable retention element configured to shift between an engaged retention position retaining the medical device with respect to the device spreader, and a disengaged position allowing the medical device to be released from the device spreader; and the control handle further includes a release system configured to shift the movable retention element from the engaged retention position to the disengaged position. In some embodiments, the release system includes a clip release slider mounted with respect to the control handle housing; the movable retention element extends from the device spreader to the clip release slider to be coupled to the clip release slider; and withdrawal of the clip release slider from the control handle housing shifts the movable retention element to the disengaged position. In some embodiments, the system further includes a safety button biased to retain the clip release slider with respect to the control handle housing.

In some embodiments, the system further includes a flexible elongate member having a distal end and a proximal end, and extending distally from the control handle through a steerable flexible elongate member. In some aspects, the device spreader is mounted on the distal end of the flexible elongate member; the proximal end of the flexible elongate member is fixed with respect to the control handle; and the control handle is movably mounted on a connecting tube to move the flexible elongate member and the device spreader axially and/or rotationally with respect to the steerable flexible elongate member.

In some embodiments, the system further includes a handle lock operably associated with the control handle to fix a position of the control handle and the device spreader with respect to the steerable flexible elongate member.

In accordance with various principles of the present disclosure, a control handle operably associated with a device spreader is configured to deliver and deploy a medical device having a first arm configured to engage a first arm of the medical device and a second arm configured to engage the second arm of the medical device. In some aspects, the control handle includes a control handle housing; and an actuator controller movable with respect to the control handle housing and including a lever handle pivotable with respect to the control handle housing to cause an actuator operably engaged with the device spreader and the actuator controller to shift the device spreader arms between closed and open configurations to shift the medical device between closed and open configurations.

In some embodiments, the control handle further includes a grasper limiter operatively associated with the control handle to limit the extent to which the lever handle shifts the device spreader to open the medical device. In some embodiments, the grasper limiter includes a limit stop movable with respect to the lever handle to limit the extent to which the lever handle is movable. In some embodiments, the grasper limiter includes an adjustment gauge movable with respect to the control handle housing to indicate the degree to which the lever handle may open the device spreader to open the medical device.

Additionally, a method of deploying a medical device having a first arm and a second arm shiftable between a delivery configuration, an open configuration allowing entry of tissue therebetween, and a closed configuration in which the medical device grasps the tissue is disclosed herein. The method includes pivoting a lever handle away from a control handle to cause the medical device to shift to the open configuration; and allowing the lever handle to return to a position along the control handle to allow the medical device to shift to the closed configuration.

In some aspects, the first and second arms of the medical device are biased towards each other to shift the medical device to a neutral configuration, and a spring assist is provided in the control handle to assist the medical device in returning to a neutral configuration.

In some aspects, the method further includes moving a clip release slider with respect to the control handle to deploy the medical device by releasing the medical device from a device spreader controlled by the control handle.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In view of the length of certain elements, some elements may be identified in more than one location for the sake of clarity. On the other hand, for purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of a deployment system and handle in accordance with various aspects of the present disclosure.

FIG. 2 illustrates a perspective view of an example of an embodiment of a leaflet clip delivery and deployment system positioned, by a delivery/deployment system formed in accordance with various principles of the present disclosure, to deploy a clip with respect to a schematic representation of a heart valve leaflet.

FIG. 3 illustrates a cross-sectional view of a leaflet clip system as in FIG. 2 and FIG. 6, along line III-III of FIG. 6.

FIG. 4 illustrates a cross-sectional view of a leaflet clip system as in FIG. 2 and FIG. 6, along line IV-IV of FIG. 6.

FIG. 5 illustrates a view similar to that of FIG. 2, but with the leaflet clip delivery and deployment system in an open configuration to catch a heart valve leaflet between the leaflet clip spreader arms and the leaflet clip arms thereof.

FIG. 6 illustrates a view similar to that of FIG. 2 and FIG. 5, but with the leaflet clip delivery and deployment system in a closed configuration grasping a heart valve leaflet between the leaflet clip spreader arms and the leaflet clip arms thereof.

FIG. 7 illustrates an enlarged isolated perspective view of a leaflet clip delivery and deployment system control handle as illustrated in FIG. 1.

FIG. 8 illustrates a perspective view similar to that of FIG. 7, but with a portion of the handle housing removed.

FIG. 9 illustrates a cross-sectional view along line IX-IX of FIG. 8.

FIG. 10 illustrates a perspective view of a leaflet clip delivery and deployment system control handle as illustrated in FIG. 7, but with a portion of the handle housing removed and with the actuator controller in a different position.

FIG. 11 illustrates an enlarged perspective view of a detail of an alternate embodiment of an element illustrated in FIGS. 8-10.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “channel” or “bore” is not limited to a circular cross-section. As used herein, a “free end” of an element is a terminal end at which such element does not extend beyond.

In accordance with various principles of the present disclosure, a delivery/deployment system is configured to deliver and/or deploy a device. In accordance with various principles of the present disclosure, the delivery/deployment system is configured to operate or otherwise to cause the device to be deployed with respect to the treatment site. In some aspects, the delivery/deployment system is configured to operate and/or includes a device delivery/deployment system configured to deliver and/or deploy a device. In some aspects, the delivery/deployment system includes a handle configured to operate such device delivery/deployment system to deliver and/or deploy the associated device. In some aspects, the device is an implantable device, and the device delivery/deployment system is configured to deliver and deploy the implantable device, such as to deploy the implantable device with respect to an anatomical site, such as a treatment site on or within a patient's body. In some embodiments, the implantable device is a clip or clamp.

In accordance with various principles of the present disclosure, a delivery/deployment system includes flexible elongate member, a handle mounted on a proximal end of a flexible elongate member, and a device delivery/deployment system mounted on the distal end of the flexible elongate member. In some embodiments, the position of the flexible elongate member may be fixable in a desired linear position and/or angular orientation with respect to the handle, such as to prevent linear and/or rotational travel of the handle.

In accordance with various principles of the present disclosure, repositioning, repair, and/or replacement of one or more leaflets of a valve and/or chordae tendinea may be desirable to treat heart disease, and may include one or more devices to be fixed to one or more leaflets of a heart valve. Such device may include a leaflet clip or clamp (hereinafter “clip” for the sake of convenience and without intent to limit). In accordance with various principles of the present disclosure, a leaflet clip delivery and deployment system may be used to deliver and deploy the leaflet clip with respect to the heart valve leaflet. The leaflet clip delivery and deployment system may include a clip spreader configured to deliver and deploy the leaflet clip, such as to clip the leaflet clip onto a heart valve leaflet. It will be appreciated that terms such as clip, attach, affix, clamp, clasp, couple, engage, attach, deploy on, grasp, hold, secure, etc. (including other grammatical forms thereof) may be used interchangeably herein, such as with reference to the interaction of the clip and tissue engaged by the clip, without intent to limit. Moreover, it will be appreciated that descriptions of elements being coupled or engaged with other elements include direct and indirect coupling or engaging.

In accordance with various principles of the present disclosure, a delivery/deployment system is configured to deliver and/or deploy a device such as a leaflet clip, and/or a leaflet clip delivery and deployment system including a leaflet clip, to a treatment site, such as a heart valve (mitral valve or tricuspid valve), and, particularly, to a leaflet thereof. In some aspects, a delivery/deployment system formed in accordance with various principles of the present disclosure is configured to operate or otherwise to control a clip delivery and deployment system such as a clip spreader to deploy a clip. More particularly, in some aspects the clip delivery and deployment system controls a leaflet clip spreader to deploy a leaflet clip with respect to a heart valve leaflet. In some aspects, a delivery/deployment system formed in accordance with various principles of the present disclosure includes a handle configured to deliver and/or deploy a clip, such as a leaflet clip with respect to a heart valve leaflet. In some aspects, the handle is configured to operate or otherwise to control a clip delivery and deployment system, such as leaflet clip spreader, to deploy a clip, such as a leaflet clip with respect to a heart valve leaflet. It will be appreciated that although reference is made herein to a leaflet clip and leaflet clip spreader, the present disclosure need not be limited to a clip and spreader specifically configured for grasping a heart valve leaflet, principles of the present disclosure being broader than the examples of embodiments described herein.

The leaflet clip may be biased into a closed configuration, such as to clamp onto tissue. The clip spreader is configured to actuate the leaflet clip into a configuration for placement with respect to a heart valve leaflet. For instance, a leaflet clip may have first and second arms coupled together via a flex zone which may bias the leaflet clip arms together. A leaflet clip delivery and deployment system formed in accordance with various principles of the present disclosure is configured to operate the leaflet clip spreader to move to an open configuration to move (e.g., spread) the first and second arms of the leaflet clip apart into an open configuration. The leaflet clip delivery and deployment system is further configured to operate the leaflet clip spreader to move (or at least to allow movement of) the first and second arms of the leaflet clip to a closed configuration clamped on a heart valve leaflet. In embodiments in which the leaflet clip arms are biased to a closed configuration, the leaflet clip delivery and deployment system allows the leaflet clip arms to return to a closed configuration, such as to clamp onto a heart valve leaflet. In some embodiments, the leaflet clip delivery and deployment system is configured to actuate the leaflet clip spreader to reopen a leaflet clip that has been released to clamp onto a heart valve leaflet before release of the leaflet clip from the leaflet clip spreader if repositioning of the leaflet clip is desired. The spreader arms of the clip spreader and the leaflet clip arms may be coupled together to move together. As such, actuation of the clip spreader arms to move apart causes the leaflet clip arms to move apart. The leaflet clip arms may be biased into a closed configuration (close to each other to clamp onto tissue) so that release of an actuating force on the clip spreader arms allows the leaflet clip arms to return to a closed position, also returning the clip spreader arms to return to a closed position. The leaflet clip delivery and deployment system may be further configured to release the leaflet clip from the clip spreader once the leaflet clip is clamped on a heart valve leaflet.

In accordance with various aspects of the present disclosure, a leaflet clip delivery and deployment system includes a handle facilitating the above-described operations. The handle may be configured to actuate the leaflet clip spreader, such as to cause movement of a spreader arm of the leaflet clip spreader, to open or close a leaflet clip to receive or clamp onto a heart valve leaflet. In some embodiments, the handle includes a lever pivotable with respect to the main body of the handle to cause the leaflet clip to open or close, such as by operating the leaflet clip spreader to open or close the leaflet clip. In some embodiments, a limiter is positioned to limit the range of motion of the lever, such as to limit the extent to which the lever opens the leaflet clip.

In accordance with an aspect of the present disclosure, a leaflet clip delivery and deployment system includes a release element configured to cause release of the leaflet clip from the leaflet clip spreader once the leaflet clip is at the deployment site, and, optionally, engaged with tissue (e.g., a heart valve leaflet) at the deployment site. More particularly, in some embodiments, a leaflet clip spreader may include a movable retention element shiftable between an engaged position engaging a leaflet clip to maintain the leaflet clip engaged with the leaflet clip spreader, and a disengaged position allowing release of the leaflet clip from the leaflet clip spreader to deploy the leaflet clip and to remove the leaflet clip spreader and the leaflet clip delivery and deployment system. The leaflet clip may be configured to be slidingly engaged with the leaflet clip spreader and readily releasable from the leaflet clip spreader once the retention element is in the disengaged position. Once the leaflet clip is positioned as desired, the clip release slider may be actuated to release the leaflet clip from the leaflet clip spreader and thus from the leaflet clip delivery/deployment system.

In accordance with various principles of the present disclosure, a delivery/deployment system is configured to shift a movable retention element between an engaged position and a disengaged position. In some embodiments, the delivery/deployment system includes a clip release element. In some embodiments, the clip release element is operatively associated with a handle of the delivery/deployment system. In some embodiments, the clip release element is a clip release slider, slidable with respect to the handle body. For instance, the clip release element may be in a rip cord configuration. A safety latch may be provided to prevent inadvertent sliding of the clip release slider.

The devices, systems, and methods of the present disclosure may be used alone or together with other devices, systems, and methods to treat heart disease. Examples of devices, systems, and methods with which embodiments of the present disclosure may be implemented include, but are not limited to, those described in U.S. Patent Application Publication US2021/0007847, titled Devices, Systems, And Methods For Clamping A Leaflet Of A Heart Valve, and published on Jan. 14, 2021; U.S. Patent Application Publication US2021/0000597, titled Devices, Systems, And Methods For Adjustably Tensioning An Artificial Chordae Tendineae Between A Leaflet And A Papillary Muscle Or Heart Wall, and published on Jan. 7, 2021; U.S. Patent Application Publication US2021/0000598, titled Devices, Systems, And Methods For Anchoring An Artificial Chordae Tendineae To A Papillary Muscle Or Heart Wall, and published on Jan. 7, 2021; U.S. Patent Application Publication US2021/0000599, titled Devices, Systems, And Methods For Artificial Chordae Tendineae, and published on Jan. 7, 2021; U.S. Patent Application Publication 2022/0096235, titled Devices, Systems, And Methods For Adjustably Tensioning Artificial Chordae Tendineae In A Heart, and published on Mar. 31, 2022; U.S. Patent Application Publication 2023/0123832, titled Devices, Systems, And Methods For Clamping A Leaflet Of A Heart Valve, and published on Apr. 20, 2023; and U.S. Patent Application Publication 2023/0149170, titled Devices, Systems, And Methods For Positioning A Leaflet Clip, and published on May 18, 2023, each of which is herein incorporated by reference in its entirety and for all purposes. Examples of devices described therein may be modified to incorporate embodiments or one or more features of the present disclosure.

It will be appreciated that a deployment system formed in accordance with various principles of the present disclosure may be used to deploy various configurations of implantable devices such as, but not limited to, various configurations of leaflet clips, the present disclosure not being limited to implantable devices such as the leaflet clips illustrated and described herein.

Various aspects of a delivery/deployment system formed in accordance with various principles of the present disclosure will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. The following description is of illustrative examples of embodiments only, and is not intended as limiting the broader aspects of the present disclosure.

It will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). Moreover, certain features in one embodiment may be used across different embodiments and are not necessarily individually labeled when appearing in different embodiments.

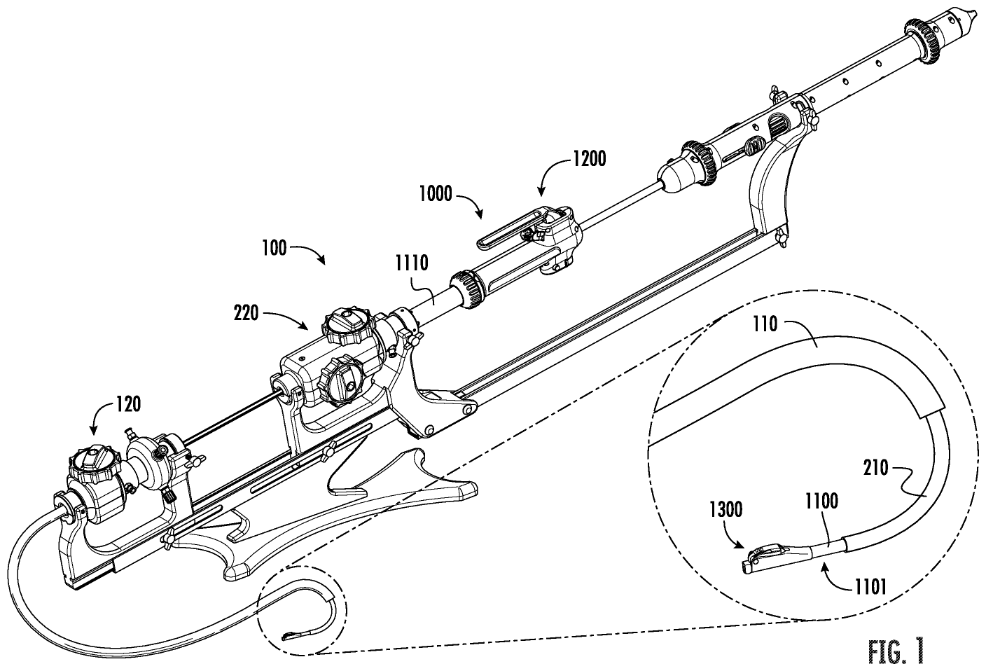

Turning now to the drawings, an example of an embodiment of a delivery/deployment system 1000 formed in accordance with various principles of the present disclosure is illustrated in FIG. 1. The delivery/deployment system 1000 optionally is part of a larger delivery/deployment system 100, such as described in further detail in in co-pending provisional patent application ______, [ATTORNEY DOCKET 2001.2787100], titled Devices, Systems, And Methods For Steering A Catheter, and filed on Jun. 21, 2022; and co-pending provisional patent application ______, [ATTORNEY DOCKET 2001.2755100], titled Devices, Systems, And Methods For Deploying An Implantable Device, and filed on Jun. 21, 2022, each of which applications is incorporated by reference herein in its entirety and for all purposes. As illustrated in the detail view, the illustrated example of an embodiment of the larger delivery/deployment system 100 includes an introducer shaft 110 (which may also be referenced as a delivery sheath, without intent to limit) configured to introduce/deliver the delivery/deployment system 100 to a treatment area. As such, the introducer shaft 110 is a tubular flexible elongate element defining a lumen therethrough sized, shaped, configured, and/or dimensioned to allow passage of further devices and/or systems therethrough for delivery to the treatment site, such as devices and/or systems delivered/deployed by the delivery/deployment system 1000. As referenced herein, a treatment area is an area on or within a patient's body at which the treatment site is located. As referenced herein, a treatment site is an anatomical site at which a procedure is to be performed with the use of systems and/or devices delivered by the delivery/deployment systems 100, 1000. It will be appreciated that reference to “at” the treatment site is intended to include at or about the vicinity of (e.g., along, adjacent, etc.) the treatment site, and is not limited to just the exact site of treatment. Moreover, it will be appreciated that reference may be made herein to a treatment site, anatomical site, delivery site, deployment site, implant/implantation site, site of implantation, target site, etc., interchangeably and without intent to limit. The introducer shaft 110 may be steerable, such as one-way or two-way steerable within a steering plane, such as with the use of a control handle 120 operatively associated therewith. The delivery/deployment system 100 may include an additional steerable flexible elongate member 210 configured to provide further steering capabilities to the delivery/deployment system 1000 to guide and direct devices and/or systems more closely to the desired treatment site. The steerable flexible elongate member 210 may be tubular, defining a lumen therethrough sized, shaped, configured, and/or dimensioned to allow passage of further devices and/or systems therethrough for delivery to the treatment site, such as devices and/or systems delivered/deployed by the delivery/deployment system 1000. The steerable flexible elongate member 210 may be steerable, such as two-way steerable within a steering plane, four-way within two transverse steering planes, etc., such as with the use of a control handle 220 operatively associated therewith.

The example of an embodiment of a delivery/deployment system 1000 illustrated in FIG. 1 includes a flexible elongate member 1100 (e.g., a delivery catheter, shaft, tube, etc.) with a control handle 1200 operatively associated therewith. The flexible elongate member 1100 is configured to deliver a device delivery/deployment system 1300 to a treatment site. The control handle 1200 is operatively associated with the flexible elongate member 1100 and/or the device delivery/deployment system 1300 to deliver the device delivery/deployment system 1300 to the treatment site and/or to operate the device delivery/deployment system 1300 to deploy a device with respect to the treatment site.

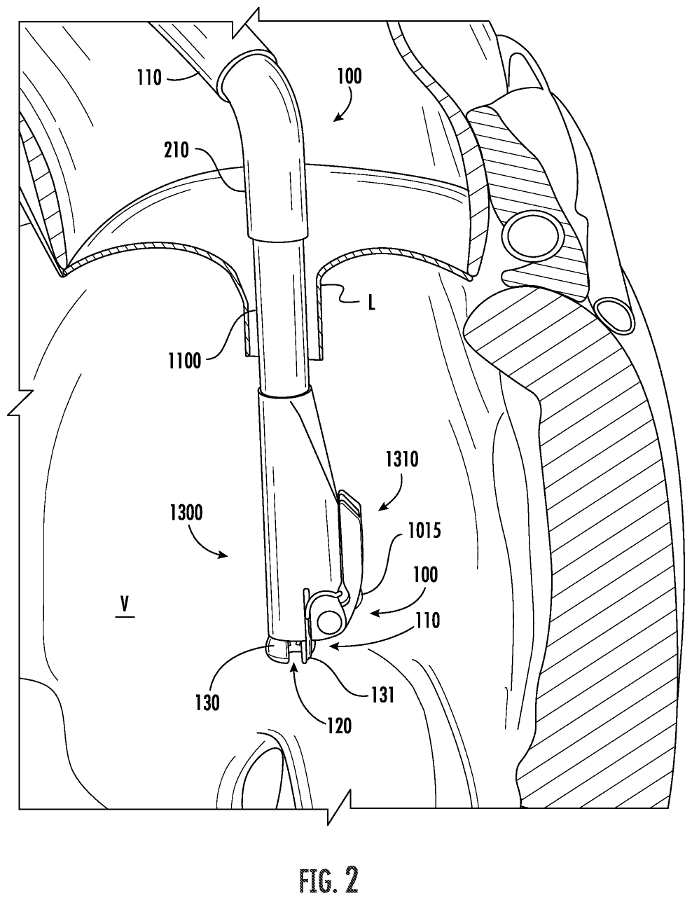

In accordance with various principles of the present disclosure, the delivery/deployment system 1000 may deliver, operate, and/or deploy cardiac repair devices and/or systems, such as heart valve leaflet repair devices and/or systems. In accordance with various further principles of the present disclosure, the delivery/deployment system 1000 may deliver, operate, and/or deploy leaflet clips and/or a delivery and deployment system 1300 for delivering/deploying leaflet clips. An example of an embodiment of a device delivery/deployment system 1300 delivered, operated, and/or deployed by the delivery/deployment system 1000 in the form of a leaflet clip delivery and deployment system 1300 is illustrated in FIG. 2 in an example of a treatment area within a heart. More particularly, an example of an embodiment of a leaflet clip delivery/deployment system 1300 is illustrated in FIG. 2 as being delivered by the delivery/deployment system 1000 though an introducer shaft 110 and a steerable flexible elongate member 210. The introducer shaft 110 delivers the leaflet clip delivery and deployment system 1300 to a treatment area, such as a heart ventricle in the example of an embodiment illustrated in FIG. 2. The steerable flexible elongate member 210 may further guide and direct the flexible elongate member 1100 of the delivery/deployment system 1000 to deliver the leaflet clip delivery and deployment system 1300 to the treatment site, such as a heart valve leaflet L in the example of an embodiment illustrated in FIG. 2. It will be appreciated that further details of an example of an embodiment of components and operation of the introducer shaft 110 and a steerable flexible elongate member 210 are provided in above-incorporated co-pending provisional patent application ______, [ATTORNEY DOCKET 2001.2787100]. However, the present disclosure need not be limited by the examples of embodiments disclosed therein.

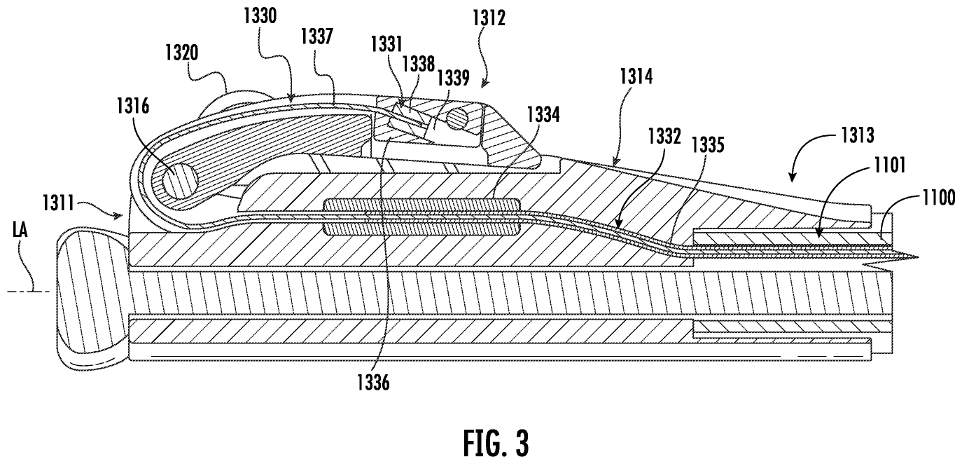

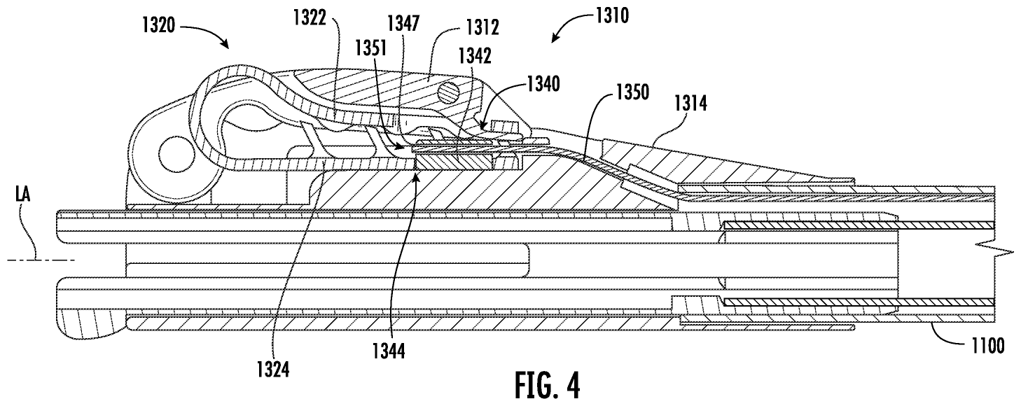

In accordance with an aspect of the present disclosure, a leaflet clip delivery and deployment system 1300 delivered by a delivery/deployment system 1000 formed in accordance with various principles of the present disclosure includes leaflet clip spreader 1310 configured to deliver and deploy a leaflet clip 1050, as illustrated in greater detail in FIG. 3 and FIG. 4. The leaflet clip delivery and deployment system 1300 may be delivered on a distal end 1101 of the flexible elongate member 1100, and the flexible elongate member 1100 may thus be referenced herein as a spreader shaft or grasper shaft 1100 without intent to limit. The leaflet clip spreader 1310 includes a ventricular spreader arm 1312 and an atrial spreader arm 1314, which may be pivotably coupled together, such as about a hinge or pivot 1316. The ventricular spreader arm 1312 is configured to engage the ventricular clip arm 1322 of the leaflet clip 1320 (the arm of the leaflet clip 1320 configured to be positioned on a side of the heart valve leaflet L facing the heart ventricle), and the atrial spreader arm 1314 is configured to engage the atrial clip arm 1324 of the leaflet clip 1320 (configured to be positioned on a side of the heart valve leaflet L facing the heart atrium). In embodiments in which the ventricular clip arm 1322 and the atrial clip arm 1324 of the leaflet clip 1320 are biased towards each other, coupling of the leaflet clip spreader 1310 with the leaflet clip 1320 may allow the leaflet clip 1320 to maintain the ventricular spreader arm 1312 and the atrial spreader arm 1314 of the leaflet clip spreader 1310 in a generally closed configuration until actuated into an open configuration.

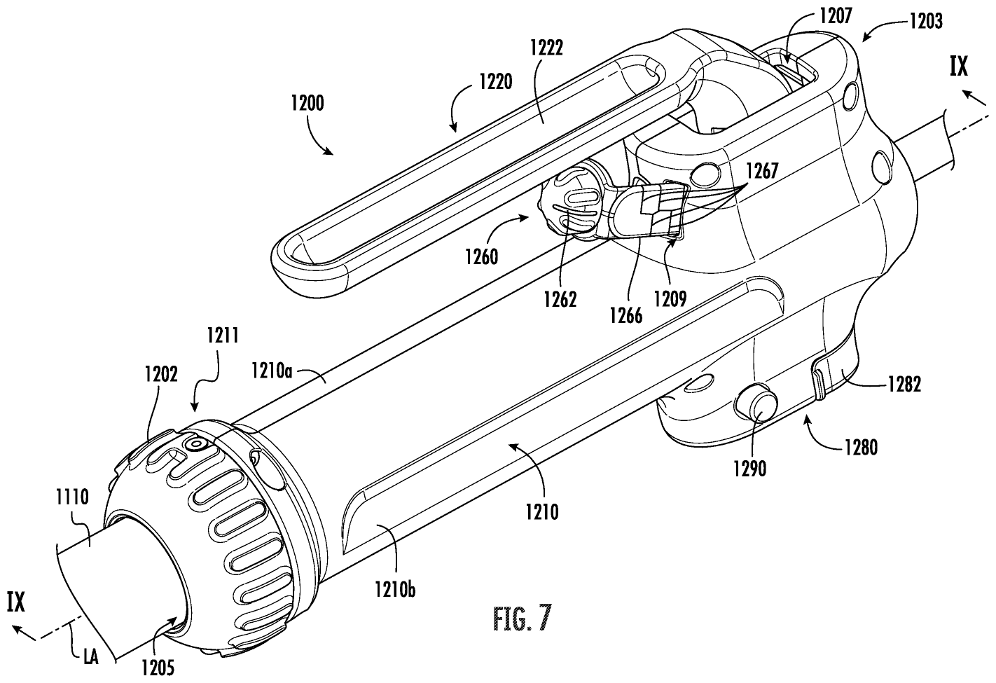

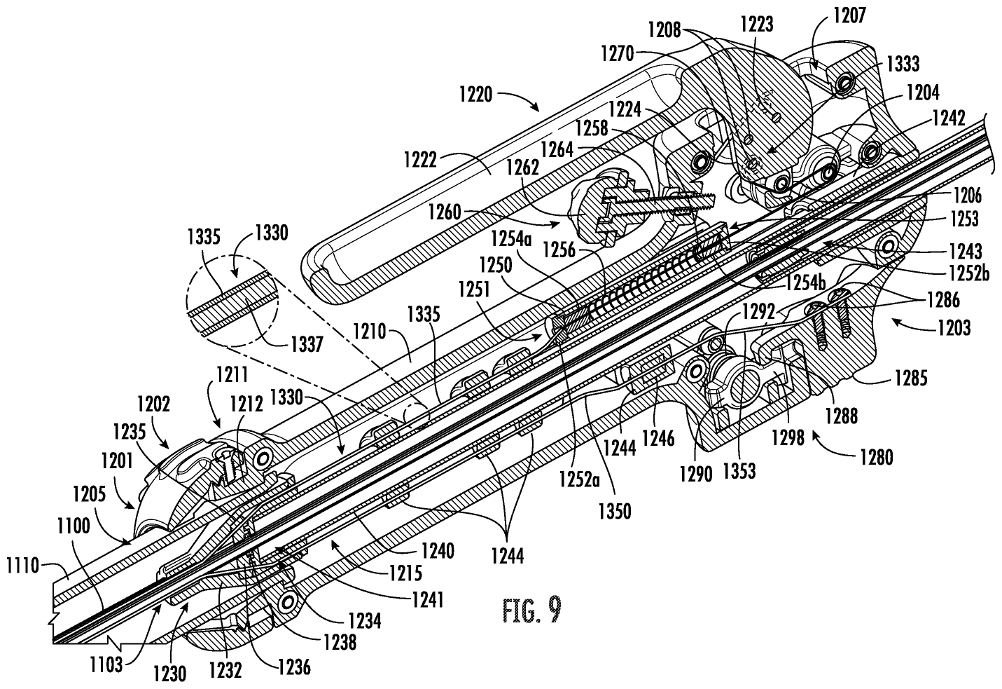

In accordance with an aspect of the present disclosure, the leaflet clip delivery and deployment system 1300 may be navigated with respect to the heart valve leaflet L by manipulation of the control handle 1200. For instance, the grasper shaft 1100 may be coupled with the control handle 1200 such that rotation and/or axial movement of the control handle 1200 causes corresponding rotation and/or axial movement of the grasper shaft 1100 (e.g., about or along the longitudinal axis LA indicated, for example, in FIG. 3 and FIG. 7). In the example of an embodiment illustrated in FIGS. 7-10, the grasper shaft 1100 is operatively coupled to the control handle 1200 via a hub system 1230, which may also serve to position and support the grasper shaft 1100 within and with respect to a connecting tube 1110. The connecting tube 1110 may be fixedly mounted with respect to the control handle 220 of the steerable flexible elongate member 210, and the control handle 1200 axially and rotationally movably mounted on the connecting tube 1110. The proximal end 1103 of the grasper shaft 1100 may be bonded within a counterbore in the adapter 1232 (such as illustrated in FIG. 9), and the adapter 1232 is coupled with the control handle housing 1210 such that movement of the control handle housing 1210 (e.g., with respect to the connecting tube 1110) moves the grasper shaft 1100. More particularly, in the example of an embodiment illustrated in FIG. 9, the adapter 1232 is bonded over a hub 1234, and the hub 1234 is bonded over a distal end 1241 of a grasper extension shaft 1240. The proximal end 1243 of the grasper extension shaft 1240 is bonded within an extension shaft hub 1242 mounted within a proximal end 1203 of the control handle housing 1210. The extension shaft hub 1242 is mounted within the control handle housing 1210, such as with a keyed connection, to be movable with the control handle housing 1210. Thus, axial or rotational movement of the control handle housing 1210 is imparted to the grasper shaft 1100 via the grasper extension shaft 1240, the hub 1234, and the adapter 1232. It will be appreciated that bonding may not be necessary to couple any or all the control handle housing 1210, the grasper extension shaft 1240, the hub 1234, the adapter 1232, and the grasper shaft 1110 so that movement of one causes movement of the other. For instance, keyed connections or other mechanical connections or otherwise which allow axial or rotational movement of one element to be imparted to the element connected thereto are within the scope and spirit of the present disclosure as may be appreciated by those of ordinary skill in the art.

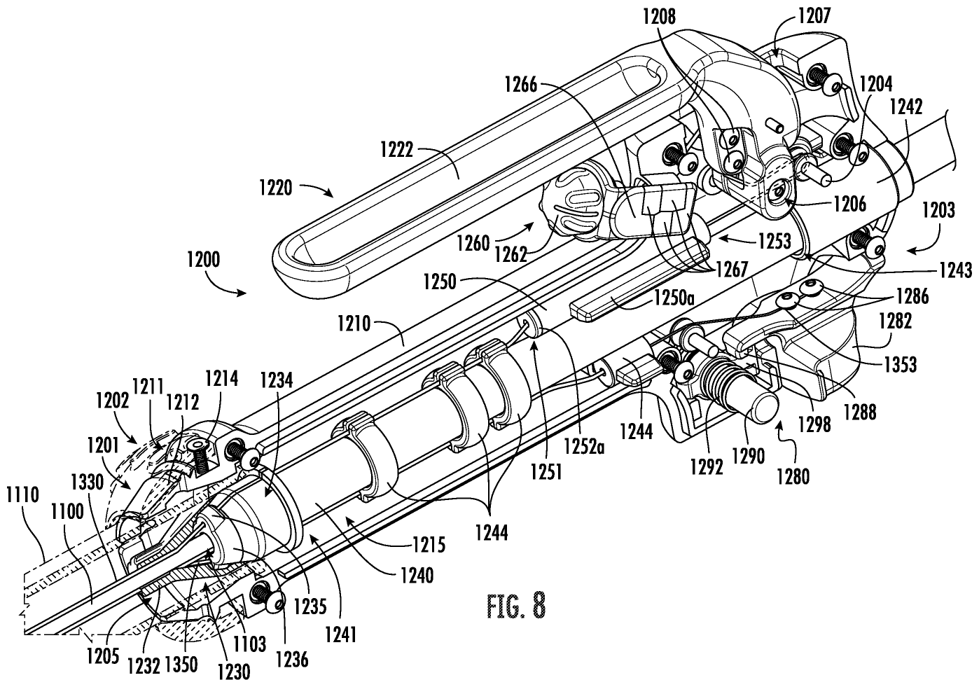

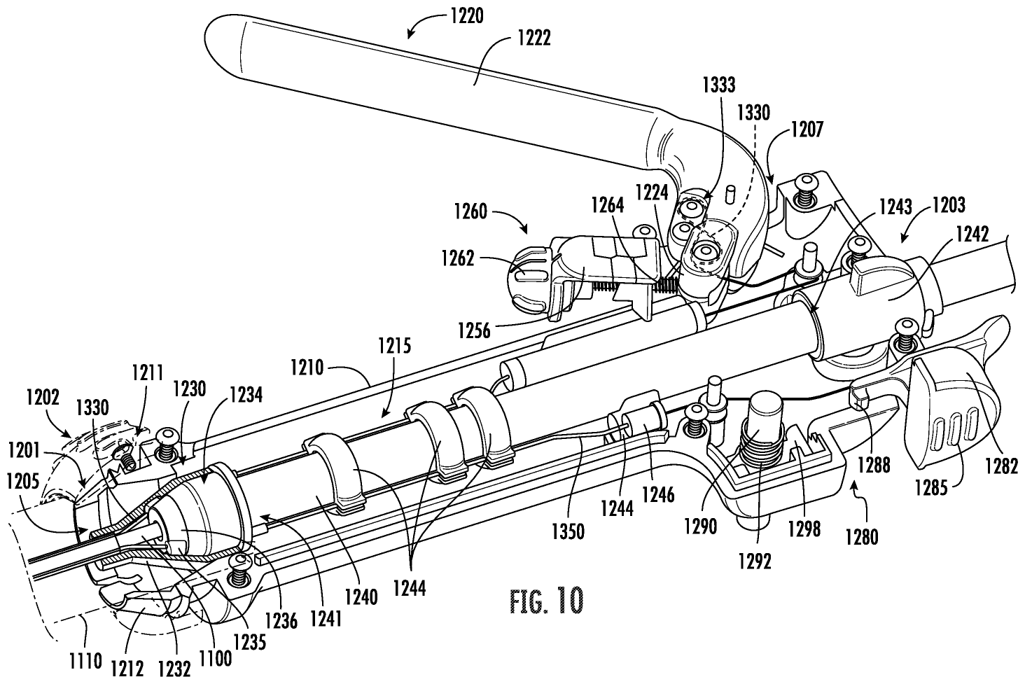

Once the desired position of the leaflet clip delivery and deployment system 1300 is achieved, then the position of the control handle 1200 may be substantially fixed or locked in place to maintain the desired position of the leaflet clip delivery and deployment system 1300. In the example of an embodiment illustrated in FIG. 7, FIG. 8, FIG. 9, and FIG. 10, a handle lock 1202 (such as a collet) may be operably associated with (e.g., provided on) a distal end 1211 of the control handle housing 1210 to fix the position of the control handle 1200 and thus the grasper shaft 1100 and the leaflet clip delivery and deployment system 1300 in a desired position. More particularly, the control handle 1200 may be mounted on a connecting tube 1110 extending between the leaflet clip delivery and deployment system control handle 1200 and the steerable flexible elongate member control handle 220 (such as illustrated in FIG. 1). The connecting tube 1110 thus may be considered a grasper telescope shaft allowing telescoping of the grasper shaft 1100 out of and into the steerable flexible elongate member 210, as well as rotation of the grasper shaft 1100 with respect to the steerable flexible elongate member 1020. The handle lock 1202 may be rotatable with respect to a distal end 1211 of the control handle housing 1210, such as to tighten grasping fingers 1212 formed on the distal end 1211 of the control handle housing 1210 onto the connecting tube 1110 to fix the control handle 1200 with respect to the connecting tube 1110. The handle lock 1202 may be threadedly engaged with the distal end 1211 of the control handle housing 1210 so that rotation in one direction tightens the grasping fingers 1212 onto the connecting tube 1110, and rotation in the opposite direction loosens the grasping fingers 1212 engagement with the connecting tube 1110 to allow movement of the control handle 1200 with respect thereto. A locking screw 1214 may maintain the handle lock 1202 on the distal end 1211 of the control handle housing 1210 so that the handle lock 1202 may move (e.g., translate distally or proximally) with respect to the control handle housing 1210 without detaching therefrom.

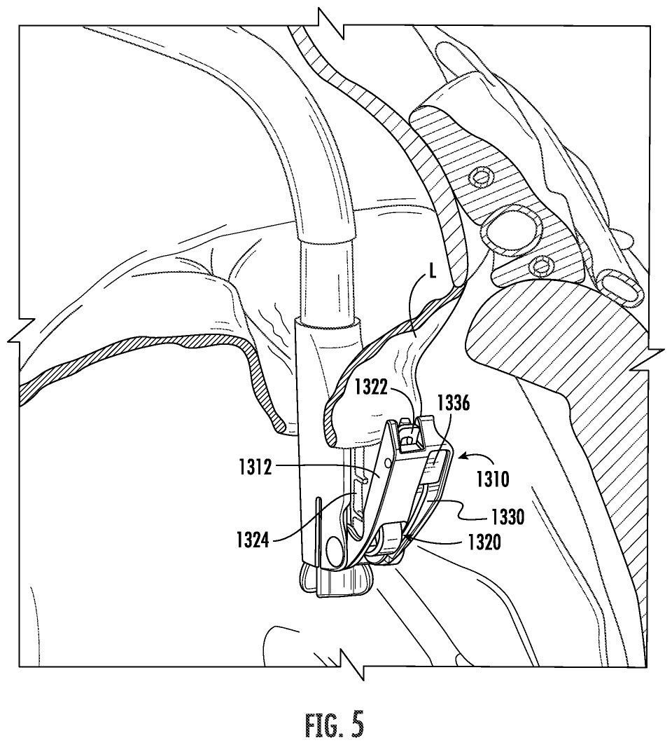

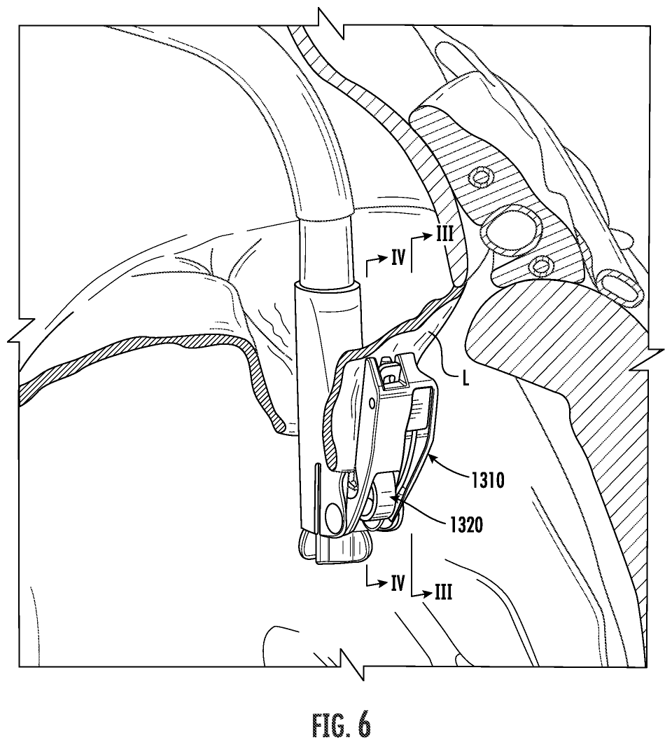

In accordance with a further aspect of the present disclosure, a leaflet clip delivery and deployment system 1300 formed in accordance with various principles of the present disclosure includes an actuator 1330 (e.g., a pull wire) operatively engaged with the leaflet clip spreader 1310 to actuate the leaflet clip spreader 1310 to shift between a delivery configuration (e.g., as illustrated in FIG. 2, FIG. 3, and FIG. 4) and an open configuration (e.g., as illustrated in FIG. 5). More particularly, when the leaflet clip 1320 is in a delivery configuration, the leaflet clip spreader arms 1312, 1314 and the leaflet clip arms 1322, 1324 are in a generally neutral position, such as a closed configuration. The actuator 1330 actuates the leaflet clip spreader 1310 to shift the leaflet clip spreader arms 1312, 1314 and the leaflet clip arms 1322, 1324 into an open position (spreading the leaflet clip spreader arms 1312, 1314 apart) to facilitate deployment of the leaflet clip 1320. In the open configuration, a heart valve leaflet L may enter between the leaflet clip arms 1322, 1324 either by the natural motion of the heart valve leaflet L (“catching” the heart valve leaflet L by allowing it to move into the space between the leaflet clip arms 1322, 1324), or by moving the leaflet clip spreader 1310 so that the leaflet clip arms 1322, 1324 surround the heart valve leaflet L. In a closed configuration, the leaflet clip 1320 is clamped on and grasping the heart valve leaflet L, such as illustrated in FIG. 6.

In some embodiments, the actuator 1330 is coupled to at least one of the arms 1312, 1314 of the leaflet clip spreader 1310, such as to move the ventricular spreader arm 1312 with respect to the atrial spreader arm 1314, such as illustrated in FIG. 3. In the illustrated example of an embodiment, the actuator 1330 includes an actuator cable 1332 having a distal end 1331 coupled to the ventricular spreader arm 1312. A proximal end 1333 actuator cable 1332 is coupled to the control handle 1200 (as shown, generally, in FIG. 8 and FIG. 9, and discussed in further detail below) to be accessible (e.g., outside the patient) by a medical professional to actuate the actuator 1330. The actuator cable 1332 may be a metal, polymer, or other suitable biocompatible material capable of withstanding the forces necessary to actuate the leaflet clip spreader 1310 as well as able to be navigated through a tortuous pathway within the patient's body. In some embodiments, the actuator cable 1332 is in the form of a Bowden cable having an outer cable sheath 1335 and an inner cable 1337 (e.g., pull wire). The outer cable sheath 1335 may extend distally from the control handle 1200 and terminate within a Bowden termination and pull cable passage 1334 mounted, for instance, with respect to the leaflet clip spreader 1310, and the inner cable 1337 may then continue distally, around the distal end 1311 of the leaflet clip spreader 1310 and around the leaflet clip 1320, to be coupled to a leaflet clip spreader arm 1312, 1314 (e.g., a ventricular spreader arm 1312) to be moved with respect to the other leaflet clip arm 1324, 1322, such as upon pulling the actuator cable 1332 (e.g., pulling the inner cable 1335 proximally). The distal end 1331 of the actuator cable 1332 may be fixed to the ventricular spreader arm 1312 such as by coupling the inner cable 1337 with a cable holder 1336 (e.g., a boss) mounted or coupled or otherwise fixed with respect to the ventricular spreader arm 1312. The distal end 1331 of the actuator cable 1332 (e.g., the inner cable 1337) may extend through a bore 1339 within the cable holder 1336 and have a widened distalmost end (e.g., the free end) to prevent the actuator cable 1332 from being withdrawn from the bore 1339 when actuating the ventricular spreader arm 1312 to open. The widened distalmost end of the actuator cable 1332 may be formed by the actuator cable 1332 itself (e.g., a knot or loop, or a crimping or widening or flattening of the actuator cable 1332), or by an element coupled thereto, such as a metal fitting 1338 (e.g., crimped collar, cylinder, bead, overmolded holder positioned over the actuator cable 1332, or a pin or other element about which the actuator cable 1332 is wrapped, etc.). It will be appreciated that other manners of coupling an element, such as an actuator cable 1332, of the actuator 1330 to the leaflet clip spreader 1310 (e.g., welding, brazing, adhering, interference fit, etc.) are within the scope and spirit of the present disclosure, the particulars of which are not critical to the general principles of the present disclosure. In some embodiments, the actuator 1330 is coupled to the leaflet clip spreader 1310 so that the distal end 1331 thereof is positioned adjacent the free end of the ventricular spreader arm 1312 and extends distally therefrom toward the pivot 1316, then around the distal end 1311 of the leaflet clip spreader 1310, and then towards the proximal end 1313 of the leaflet clip spreader 1310 and proximally towards the control handle 1200. It may be appreciated that such configuration generally allows for the optimal force vector to be applied to actuate the leaflet clip spreader 1310.

The actuator 1330 extends proximally, such as within the grasper shaft 1100, from the leaflet clip delivery and deployment system 1300 (e.g., as illustrated in FIG. 3) to the control handle 1200 of the leaflet clip delivery and deployment system 1000 illustrated in FIG. 1. As may be appreciated more readily with reference to the enlarged view of FIG. 7, the control handle 1200 includes a main body or housing 1210 with an actuator controller 1220 operatively associated therewith. The control handle housing 1210 may be formed of two housing sections or halves 1210a, 1210b coupled together to form the control handle housing 1210 and to define an interior 1215 illustrated in further detail in FIGS. 8-10. In the examples of embodiments illustrated in FIGS. 8-10, the actuator 1330 extends into the interior 1215 of the control handle housing 1210 (e.g., generally through an opening 1205 in the distal end 1201 of the control handle 1200) to be operatively engaged with the actuator controller 1220 operatively associated with the control handle 1200. As illustrated, for example, in FIG. 8, FIG. 9, and FIG. 10, the actuator 1330 may extend through the hub system 1230 within the distal end 1211 of the control handle housing 1210. More particularly, the hub 1234 of the hub system 1230 may include an actuator guide 1235 through which the actuator 1330 is guided. In some embodiments, the actuator guide 1235 is mounted on a cap 1236 at a distal end of the hub 1234. The hub 1234 may also house a gasket 1238 to seal against additional components of the larger delivery/deployment system 100 extending proximally from the grasper shaft 1110 and the hub system 1230 into the interior 1215 of the control handle housing 1210 and proximally thereto. The gasket 1238 may thereby seal the interior 1215 of the housing 1210 from bodily fluids and/or saline or other fluids which may be within the grasper shaft 1110. In some embodiments, the gasket 1238 is housed between the main body of the hub 1234 and the cap 1236, such as in the example of an embodiment illustrated in FIG. 8. Additional guide rings 1244 may be mounted along the grasper shaft extension 1240 to further guide elements such as the actuator 1330 within the interior 1215 of the control handle housing 1210.

Additional gaskets may be provided to guide, seal, etc., various elements within the interior 1215 of the control handle housing 1210. For instance, the actuator 1330 may extend through an actuator guide housing 1250 supported within the control handle housing 1210 such as via engagement of housing extensions 1250a (e.g., wings) with the interior 1215 of the control handle housing 1210 (e.g., a support element formed therein). In an embodiment having an actuator 1330 formed from a two-part actuator cable 1332, the outer sheath 1335 of the actuator cable 1332 may terminate at a distal cap 1252a mounted at a distal end 1251 of the actuator guide housing 1250. The inner cable 1337 extends through the actuator guide housing 1250 to the proximal end 1253 thereof and through a proximal sealing cap 1252b mounted on the proximal end 1253 of the actuator guide housing 1250. The proximal sealing cap 1252b may serve as a seal or gasket for the actuator cable 1332 to seal the inner cable 1337, such as from any bodily fluid within the interior 1215 of the control handle housing 1210.

The actuator 1330 may be operatively coupled with the actuator controller 1220 in any of a variety of manners such that movement of the actuator controller 1220, such as via movement of the lever handle 1222, causes movement of the actuator 1330 to actuate the leaflet clip delivery and deployment system 1300. In the examples of embodiments illustrated in FIG. 8, FIG. 9, and FIG. 10, the actuator 1330 extends proximally towards the proximal end 1203 of the housing 1210 and loops around a proximal pulley 1204 to extend distally to loop around a distal pulley 1206 and then towards a fixation location on the actuator controller 1220. The proximal end 1333 of the actuator 1330 is fixed with respect to the actuator controller 1220, such as via one or more fixation posts or screws 1208. In the example of an embodiment illustrated in FIG. 9, the proximal end 1333 of the actuator 1330 may be looped around two fixation posts 1208 to secure the actuator 1330 to the actuator controller 1220. As may be appreciated with reference to FIG. 10, movement of the actuator controller 1220, such as by pivoting the lever handle 1222, with respect to the handle housing 1210 pulls the actuator 1330 proximally. And, as may be appreciated with reference FIG. 3, proximal pulling on the actuator 1330 causes the spreader arms 1312, 1314 of the leaflet clip spreader 1310 to open, such as by pulling the ventricular spreader arm 1312 away from the atrial spreader arm 1314 and opening the leaflet clip spreader 1310 such as illustrated in FIG. 5. Finally, it may be appreciated that the pulleys 1204, 1206 may not only facilitate translation of the actuator 1330 but may also align the actuator 1330 with the grasper shaft 1100 as the actuator 1330 transitions between the actuator controller 1220 and the grasper shaft 1100.

More particularly, in the example of an embodiment illustrated in FIG. 7, FIG. 8, FIG. 9, and FIG. 10, the actuator controller 1220 includes a lever handle 1222 movably associated with and extending generally alongside (e.g., generally parallel to the longitudinal axis LA of) the control handle housing 1210. As may be appreciated with reference to FIG. 7, FIG. 8, FIG. 9, and FIG. 10, the illustrated example of an embodiment of a lever handle 1222 is pivotably coupled with respect to a proximal end 1203 of the control handle housing 1210 (such as via a lever pivot pin 1223), extends through a lever opening 1207 through the control handle housing 1210, and extends distally towards the distal end 1211 of the control handle housing 1210. However, it will be appreciated that other configurations are within the scope and spirit of the present disclosure. In some aspects, the lever handle 1222 is pivoted away from the control handle housing 1210 to actuate the actuator controller 1220 to actuate the actuator 1330 to open the leaflet clip 1320. Likewise, in some aspects, pivoting of the lever handle 1222 towards the control handle housing 1210 allows the leaflet clip 1320 to close, such as by actuating the actuator controller 1220 to actuate the actuator 1330 to close the leaflet clip 1320. Such movement of the lever handle 1222 may be considered to correspond to the associated opening and closing movement of the leaflet clip 1330.

Various features and structures may be provided to regulate or modify or adjust movement of the actuator controller 1220, such as to regulate or modify or adjust the actuator 1330 and the leaflet clip delivery and deployment system 1300. For instance, in some embodiments a grasper limiter 1260 is provided with respect to the control handle housing 1210 and the lever handle 1222 to limit the extent to which the actuator controller 1220 may be opened and thereby to limit the amount the leaflet clip 1320 may be opened. The example of an embodiment of a grasper limiter 1260 illustrated in FIGS. 8-10 includes a grasper limiter control knob 1262 which advances or retracts a limit stop 1264 with respect to a shoulder stop 1224 on the actuator controller 1220. As the lever handle 1222 is moved with respect to the control handle housing 1210, the limit stop 1264 moves (e.g., pivots, rotates, swings, etc.) towards the grasper limiter 1260. Contact of the shoulder stop 1224 with the limit stop 1264 limits the movement of the actuator controller 1220 and thereby limits linear travel of the actuator 1330. By limiting movement of the actuator 1330, the grasper limiter 1260 limits the extent to which the leaflet clip spreader 1310 and the leaflet clip 1320 are opened, and determines the angle to which the leaflet clip 1320 opens. The grasper limiter 1260 may thus be considered an angle set feature correlating movement of the actuator controller 1220, such as the opening or pivoting of the lever handle 1222, with how far the leaflet clip spreader 1310 opens the leaflet clip 1320. The grasper limiter 1260 may include an adjustment gauge 1266 operatively associated with (e.g., mounted with respect to) the grasper limiter control knob 1262 and having indicators 1267 representing the extent to which the leaflet clip 1320 is opened by operation of the lever handle 1222. More particularly, movement (e.g., rotation) of the grasper limiter control knob 1262 advances or retracts the grasper limiter control knob 1262, the limit stop 1264, and the adjustment gauge 1266 with respect to the control handle housing 1210 and the shoulder stop 1224 on the lever handle 1222. With movement of the grasper limiter control knob 1262, the adjustment gauge 1266 extends into or advances out of an opening 1209 in the control handle housing 1210. The position of the indicators 1257 on the adjustment gauge 1266 with respect to the control handle housing 1210 provide an indication of the extent to which the leaflet clip 1320 is open. As used in a heart valve repair procedure, the leaflet clip arms 1322, 1324 may be opened approximately 45° or approximately 60° with respect to each other, such as to catch a heart valve leaflet L therebetween. The leaflet clip arms 1322, 1324 may be opened further, such as up to approximately 135°, such as to adjust the position of the leaflet clip 1320 with respect to the heart valve leaflet L or one or more chordae tendineae. If the leaflet clip 1320 becomes tangled with one or more chordae tendineae, the leaflet clip arms 1322, 1324 may be opened to approximately 180° to be more readily removed from the treatment area (which may be referenced as “bail out”). Such opening of the leaflet clip 1320 may exceed the strain rate of the leaflet clip 1320 and thus deform and destroy the leaflet clip 1320 for further use. but extend (exceed strain rate). The indicators 1267 may be advantageously used to control opening of the leaflet clip arms 1322, 1324 as desired.

If the leaflet clip arms 1322, 1324 are biased into a closed configuration with respect to each other to clamp an element, such as tissue (e.g., a heart valve leaflet L), therebetween, then the leaflet clip arms 1322, 1324 simply may be allowed to close, such as by removing an opening force on the actuator 1330 (e.g., by ceasing to pull distally on the actuator 1330). In some embodiments, the lever handle 1222 may be biased to return to a configuration (e.g., as illustrated in FIG. 7, FIG. 8, and FIG. 9) which does not exert a proximal pulling force on the actuator 1330. For instance, in some embodiments, a spring such as a torsion spring 1270 returns the lever handle 1222 to a neutral position not actuating the actuator 1330, such as to allow the leaflet clip 1320 to return to a neutral position (e.g., a closed position as illustrated, for example, in FIG. 3). The torsion spring 1270 thereby acts as a spring assist to allow the leaflet clip 1320 to return to a neutral configuration, such as a closed configuration, when an opening force is no longer applied to the lever handle 1222.

In some embodiments, one or more assists are provided to mitigate the effects forces such as friction (such as within the systems 100, 1000) may have on a previous pulling force on the actuator 1330 to allow the actuator 1330 to allow the leaflet clip 1320 to return to a neutral closed configuration (e.g., clamping on the heart valve leaflet L) once forces on the actuator 1330 have been released. For instance, in the example of an embodiment illustrated in FIG. 9, the actuator guide housing 1250 may exert frictional forces on the actuator 1330, such as via one or both of the caps 1252a, 1252b thereof, and/or gaskets within the actuator guide housing 1250. For instance, in some embodiments, one or more additional gaskets may be provided within the actuator guide housing 1250. In the example of an embodiment illustrated in FIG. 9, the outer sheath 1335 of the actuator 1330 is bonded to the distal sealing cap 1252a, and the inner cable 1337 extends through and is bonded to a spring plate 1254a adjacent and proximal to the distal sealing cap 1252a. The inner cable 1337 extends proximally through the actuator guide housing 1250 and optionally also through a proximal gasket 1254b adjacent and distal to the proximal sealing cap 1252b. The illustrated actuator guide housing 1250 includes a spring 1256 operatively associated with the actuator 1330 to assist the actuator 1330 in moving distally with respect to the actuator guide housing 1250. More particularly, the spring 1256 is compressed upon actuating the actuator controller 1220 to pull the actuator 1330 proximally to open the leaflet clip spreader 1310, pulling the spring plate 1254a proximally against the spring 1256 to compress the spring 1256 between the spring plate 1254a and the proximal gasket 1254b. When the actuator controller 1220 is no longer actuated, the spring 1256 returns the spring plate 1254a to a distal location adjacent the distal sealing cap 1252a, thereby pulling/returning the actuator 1330 to its original position (prior to actuation of the actuator controller 1220) to assist in returning the leaflet clip 1230 to a neutral (e.g., closed) configuration. The proximal end of the spring 1258 may abut and/or push off a spring washer 1258 positioned between the spring 1258 and the proximal gasket 1254b to protect the proximal gasket 1254b

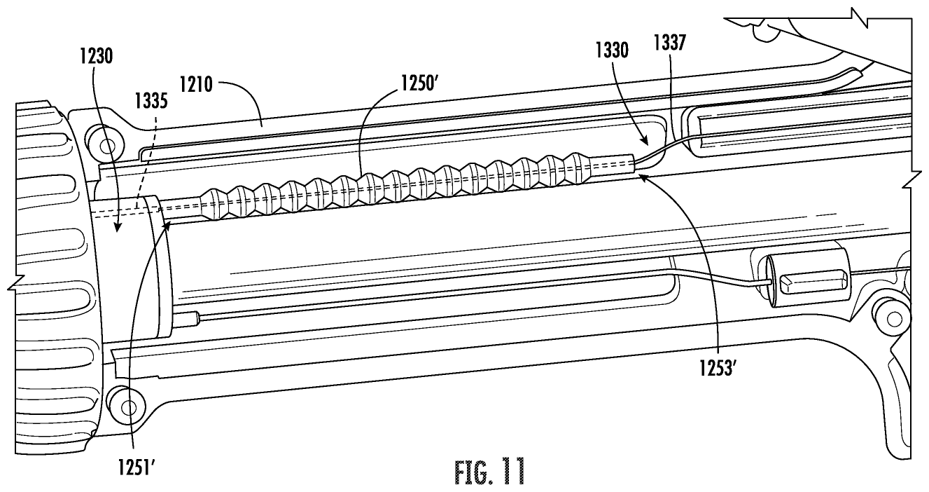

Alternatively, in the example of an embodiment illustrated in FIG. 11, the actuator guide housing 1250 may be resilient, e.g., an elastic, stretchable, expandable, etc., actuator guide housing 1250′. The distal end 1251′ of the actuator guide housing 1250′ may be bonded to the hub assembly 1230, and the proximal end 1253′ of the actuator guide housing 1250′ may be bonded to the inner cable 1337 of the actuator 1330 (with the outer sheath 1335 of the actuator 1330 ending within the hub assembly 1230). The actuator guide housing 1250′ thus stretches with proximal movement of the actuator 1330 upon actuation of the actuator controller 1220. Once the actuator controller 1220 is no longer actuated, the expanded actuator guide housing 1250′ returns to its neutral configuration, returning the actuator 1330 to its original position to assist in returning the leaflet clip 1320 to a neutral (e.g., closed) configuration. Optionally, the actuator guide housing 1250′ is a sealed pull wire gasket coupled (e.g., bonded) to the actuator 1330 (e.g., the inner cable 1337) to eliminate the need for additional gaskets to seal around (and potentially add friction to sliding of) a moving cable/pull wire such as the actuator 1330.

If for any reason it is desired or medically indicated to adjust the position of the leaflet clip 1320 (e.g., if a different position or orientation of the leaflet clip 1320 is desired and/or medically indicated, if any component of the leaflet clip delivery and deployment system 1000 becomes caught on a chordae tendineae, etc.), the leaflet clip spreader 1310 may be actuated again to release the leaflet clip 1320 and the leaflet clip delivery and deployment system 1000 may be shifted to the desired position for deployment of the leaflet clip 1320.

After the leaflet clip 1320 has been closed on tissue, such as a heart valve leaflet L (as illustrated in FIG. 6), and is in the desired and/or medically indicated deployment position, the leaflet clip spreader 1310 may be removed to leave the leaflet clip 1320 in place in the deployed position. Optionally, the leaflet clip 1320 is coupled with an artificial chordae tendineae (in any manner known to those of ordinary skill in the art) which extends therefrom to be anchored at another location, such as to papillary muscle of the heart ventricle. The artificial chordae tendineae anchor and associated delivery/deployment system may be any as known to those of ordinary skill in the art, or as described in the above-incorporated U.S. Patent Application Publication 2023/0123832 titled Devices, Systems, And Methods For Clamping A Leaflet Of A Heart Valve, and published on Apr. 20, 2023; and provisional patent application ______, [ATTORNEY DOCKET 2001.2755100], filed Jun. 21, 2022. It will be appreciated that the artificial chordae tendineae may be coupled to the leaflet clip 1320 during delivery and deployment of the leaflet clip 1320 and may extend out a side of the leaflet clip spreader 1310 and towards and coupled to an artificial chordae tendineae anchor carried within the leaflet clip spreader 1310 (e.g., as illustrated in FIG. 3 and FIG. 4).

It will be appreciated that it is generally desirable to maintain the leaflet clip spreader 1310 operatively associated with the leaflet clip 1320 until the leaflet clip 1320 and any associated anchor are fully deployed. In some embodiments, a retention assembly 1340 is operatively associated with the leaflet clip spreader 1310 and the leaflet clip 1320 to retain the leaflet clip spreader 1310 with the leaflet clip 1320 together and to avoid inadvertent separation thereof. In one embodiment, such as illustrated in FIG. 4, the atrial spreader arm 1314 of the leaflet clip spreader 1310 includes an atrial spreader arm retention element 1342 in the form of a projection, and the atrial clip arm 1324 of the leaflet clip 1320 includes a mating atrial leaflet clip arm retention element 1344 in the form of a detent or aperture. In some embodiments, the atrial spreader arm retention element 1342 is in the form of a boss insertable into an atrial leaflet clip arm retention element 1344 in the form of an aperture. A movable retention element 1350 may retain the atrial spreader arm retention element 1342 and the atrial leaflet clip arm retention element 1344 in an engaged retention position (as illustrated in FIG. 3). For instance, the movable retention element 1350 may be a wire with a distal end 1351 insertable through an aperture 1347 through the atrial spreader arm retention element 1342. In such configuration, the movable retention element 1350 prevents or inhibits movement of the atrial clip arm 1324 of the leaflet clip 1320 with respect to the leaflet clip spreader 1310. The movable retention element 1350 may be shifted into a disengaged position to allow relative movement of the leaflet clip 1320 and the leaflet clip spreader 1310. For instance, the movable retention element 1350 may be pulled (e.g., proximally) out of the aperture 1347 in the atrial spreader arm retention element 1342 to allow the leaflet clip 1320 to separate from the leaflet clip spreader 1310. In some embodiments, the movable retention element 1350 is in the form of a wire, a filament, a tether, a cable (such as a Bowden cable), or the like which may be moved proximally to withdraw from an atrial leaflet clip arm retention element 1344 in the form of a boss, thereby unblocking the leaflet clip 1320 from movement with respect to the leaflet clip spreader 1310. The movement between the atrial spreader arm 1314 and the atrial clip arm 1324 may be a generally sliding movement, and may not include further movements in other directions.

In accordance with various principles of the present disclosure, the control handle 1200 of the delivery/deployment system 1000 includes a release system 1280, such as illustrated in FIG. 7, to which the movable retention element 1350 is operatively coupled. Operation of the release system 1280 operates the movable retention element 1350 to release the leaflet clip 1320 from the leaflet clip spreader 1310. More particularly, the release system 1280 of the examples of embodiments illustrated in FIGS. 7-10 includes a clip release slider 1282 operable to shift the movable retention element 1350 from an engaged to a disengaged position. Optionally, the clip release slider 1282 includes finger-gripping formations 1285 (e.g., ridges or other friction-reducing formations, such as illustrated in FIG. 9 and FIG. 10) to facilitate gripping of the clip release slider 1282 to move the clip release slider 1282 relative to the control handle housing 1210 to actuate the movable retention element 1350 to release a leaflet clip 1320 from a leaflet clip spreader 1310. The movable retention element 1350 may extend proximally from the leaflet clip spreader 1310 towards the control handle 1200 and into the control handle housing 1210 (such as through the opening 1205 in the distal end 1201 of the control handle 1200) to be operatively engaged with a clip release slider 1282 of the release system 1280. In the example of an embodiment illustrated in FIG. 8, FIG. 9, and FIG. 10, the movable retention element 1350 may extend through a gasket housing 1244 and a gasket 1246 within the gasket housing 1244. In some embodiments, the movable retention element 1350 may be formed a two part actuator cable similar to the actuator 1330 described above, with an outer cable sheath ending at the distal entrance into the gasket housing 1244 and the inner cable extending through the gasket 1246 and out of the gasket housing 1244. The proximal end 1353 of the movable retention element 1350, such as the proximal end of an inner cable thereof, may be fixed with respect to the clip release slider 1282, such as via one or more fixation posts or screws 1286, such as in the example of an embodiment illustrated in FIG. 8 and FIG. 9. Proximal movement of the clip release slider 1282 causes proximal movement of the movable retention element 1350 to shift to a disengaged configuration.

Such release of the leaflet clip 1320 from the leaflet clip spreader 1310 upon shifting of the movable retention element 1350 to a disengaged position may be a one-way release which is generally not reversable. In order to prevent inadvertent release of the leaflet clip 1320, a safety button 1290 may be provided, as in the example of an embodiment illustrated in FIG. 7. The safety button 1290 may be operatively engaged with the clip release slider 1282 to hold the clip release slider 1282 in a position retaining the movable retention element 1350 in an engaged position to hold the leaflet clip 1320 engaged with the leaflet clip spreader 1310. Intentional operation of the safety button 1290, such as shifting of the safety button 1290, disengages the safety button 1290 and the clip release slider 1282 to allow the clip release slider 1282 to be moved to proximally retract the movable retention element 1350 to shift the movable retention element 1350 into a disengaged configuration to release the leaflet clip 1320 from the leaflet clip spreader 1310. In the example of an embodiment illustrated in FIG. 8, FIG. 9, and FIG. 10, the safety button 1290 includes a biasing element 1292, such as a coil spring, to retain the safety button 1290 in an engaged, locking configuration. The safety button 1290 and the clip release slider 1282 may be engaged into a locked configuration by engagement of respective locking elements 1288, 1298 on the clip release slider 1282 and the safety button 1290 respectively. In the example of an embodiment illustrated in FIG. 8, FIG. 9, and FIG. 10, the locking elements 1288, 1298 are interlocking projections or teeth or a projection and receiving slot. Lateral shifting of the safety button 1290, against the biasing force of the biasing element 1292, releases the interlocking engagement of the locking elements 1288, 1298 to allow the locking elements 1288, 1298 to disengage so that the clip release slider 1282 may be moved to shift the movable retention element 1350 into a release position.

Although a leaflet clip delivery and deployment system 1000 is described herein as being delivered to a heart valve as a treatment site (as illustrated in FIG. 2), principles of the present disclosure may be applied to the tricuspid valve, or other anatomical sites. Moreover, principles of the present disclosure may be applied to clips or grasping elements other than those configured for use on a heart valve leaflet. For instance, principles of the present disclosure may be applied to actuate other devices which have movable elements, such as elements which are movable with respect to each other (apart from and/or closer to each other), and/or which are biased into at least one of a closed or open position and moved into the other of an open or closed position and allowed to return to the position in which the device is biased to return.

In view of the above, it should be understood that the various embodiments illustrated in the figures have several separate and independent features, which each, at least alone, has unique benefits which are desirable for, yet not critical to, the presently disclosed leaflet clip delivery and deployment device, system, and method. Therefore, the various separate features and structures described herein need not all be present in order to achieve at least some of the desired characteristics and/or benefits described herein. Only one of the various features or structures may be present to achieve a desired characteristic and/or benefit described herein. Alternatively, one or more of the features or structures described with reference to one embodiment can be combined with one or more of the features or structures of any of the other embodiments provided herein. That is, any of the features described herein can be mixed and matched to create hybrid designs, and such hybrid designs are within the scope of the present disclosure. Moreover, throughout the present disclosure, reference numbers are used to indicate a generic element or feature of the disclosed embodiment. The same or similar reference number may be used to indicate elements or features that are not identical in form, shape, structure, etc., yet which provide similar functions or benefits. Additional reference characters (such as letters, as opposed to numbers) may be used to differentiate similar elements or features from one another.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A delivery/deployment system for delivering and/or deploying a medical device having a first arm and a second arm shiftable between a closed configuration with the arms adjacent each other and an open configuration with the arms shifted apart from each other, said system comprising:
a control handle having a control handle housing and an actuator controller movable with respect to said control handle housing;
a device spreader configured to deliver and deploy the medical device and having a first arm configured to engage a first arm of the medical device and a second arm configured to engage the second arm of the medical device; and
an actuator coupled to said spreader device and extending to and coupled to said actuator controller;
wherein:
said actuator controller includes a lever handle pivotable with respect to said control handle housing to cause said actuator to shift said device spreader arms between
closed and open configurations to shift the medical device between closed and open configurations.

2. The system of claim 1, wherein said actuator is coupled to said first arm of said device spreader and extends distally therefrom and then proximally to said actuator controller, and is guided around pulleys to be coupled with said lever handle.

3. The system of claim 1, further comprising a grasper limiter operatively associated with said control handle to limit the extent to which said device spreader opens the medical device.

4. The system of claim 3, wherein said grasper limiter includes a limit stop movable with respect to said lever handle to limit the extent to which said lever handle is movable to cause said actuator to shift said device spreader to an open configuration.

5. The system of claim 4, wherein said grasper limiter includes an adjustment gauge movable with respect to said control handle housing to indicate the degree to which said lever handle may open said device spreader to open the medical device.

6. The system of claim 1, wherein said actuator controller shifts said actuator from a neutral position to an actuated position, said system further comprising a spring assist within said control handle housing configured to assist said actuator to return to the neutral position upon releasing said lever handle.

7. The system of claim 6, further comprising an actuator guide housing through which said actuator passes to extend to said actuator controller, wherein:
said spring assist comprises a spring and a spring plate within said actuator guide housing;
said spring plate is coupled with respect to said actuator; and
said spring biases said spring plate to return said actuator to the neutral position.

8. The system of claim 6, wherein said spring assist comprises a resilient actuator guide housing configured to return said actuator to the neutral position.

9. The system of claim 1, wherein:
the device spreader comprises a movable retention element configured to shift between an engaged retention position retaining the medical device with respect to said device spreader, and a disengaged position allowing the medical device to be released from said device spreader; and
said control handle further comprising a release system configured to shift said movable retention element from the engaged retention position to the disengaged position.

10. The system of claim 9, wherein:
said release system comprises a clip release slider mounted with respect to said control handle housing;
said movable retention element extends from said device spreader to said clip release slider to be coupled to said clip release slider; and
withdrawal of said clip release slider from said control handle housing shifts said movable retention element to the disengaged position.

11. The system of claim 10, further comprising a safety button biased to retain said clip release slider with respect to said control handle housing.

12. The system of claim 1, further comprising a flexible elongate member having a distal end and a proximal end, and extending distally from said control handle through a steerable flexible elongate member, wherein:
said device spreader is mounted on the distal end of said flexible elongate member;
the proximal end of said flexible elongate member is fixed with respect to said control handle; and
said control handle is movably mounted on a connecting tube to move said flexible elongate member and said device spreader axially and/or rotationally with respect to the steerable flexible elongate member.

13. The system of claim 1, further comprising a handle lock operably associated with said control handle to fix a position of said control handle and said device spreader with respect to the steerable flexible elongate member.

14. A control handle operably associated with a device spreader configured to deliver and deploy a medical device having a first arm configured to engage a first arm of the medical device and a second arm configured to engage the second arm of the medical device, said control handle comprising:
a control handle housing; and
an actuator controller movable with respect to said control handle housing and including a lever handle pivotable with respect to said control handle housing to cause an actuator operably engaged with the device spreader and said actuator controller to shift said device spreader arms between closed and open configurations to shift the medical device between closed and open configurations.

15. The control handle of claim 14, further comprising a grasper limiter operatively associated with said control handle to limit the extent to which the lever handle shifts the device spreader to open the medical device.

16. The control handle of claim 15, wherein said grasper limiter includes a limit stop movable with respect to said lever handle to limit the extent to which said lever handle is movable.

17. The control handle of claim 16, wherein said grasper limiter includes an adjustment gauge movable with respect to said control handle housing to indicate the degree to which said lever handle may open the device spreader to open the medical device.

18. A method of deploying a medical device having a first arm and a second arm shiftable between a delivery configuration, an open configuration allowing entry of tissue therebetween, and a closed configuration in which the medical device grasps the tissue, said method comprising:
pivoting a lever handle away from a control handle to cause the medical device to shift to the open configuration; and
allowing the lever handle to return to a position along the control handle to allow the medical device to shift to the closed configuration.

19. The method of claim 18, wherein the first and second arms of the medical device are biased towards each other to shift the medical device to a neutral configuration, and a spring assist is provided in the control handle to assist the medical device in returning to a neutral configuration.

20. The method of claim 18, further comprising moving a clip release slider with respect to the control handle to deploy the medical device by releasing the medical device from a device spreader controlled by the control handle.
